(19) United States
(12) Patent Application Publication
(10) Pub. No.: US 2011/0117074 A1
(43) Pub. Date: May 19, 2011

(54) METHODS OF TREATING HIV AND INFLUENZA INFECTIONS USING ASPERGILLUS ORYZAE PROTEASE

(76) Inventor: Joseph A. Cerceo, Elkton, MD (US)

(21) Appl. No.: 12/950,209

(22) Filed: Nov. 19, 2010

Related U.S. Application Data
(60) Provisional application No. 61/262,791, filed on Nov. 19, 2009.

(51) Int. Cl. A61K 38/48; A61P 31/18; A61P 31/16

(52) U.S. Cl. 424/94.63

(57) ABSTRACT

The invention provides methods for treating viral diseases such as HIV infection and influenza infection in a subject in need of treatment, comprising orally administering to the subject a composition comprising a therapeutically effective amount of an Aspergillus oryzae protease and potato dextrin as a resistant starch colloidal carrier.

### CROSS-REFERENCE TO RELATED APPLICATIONS

This application claims the benefit of U.S. Appl. No. 61/262,791, filed Nov. 19, 2009. The content of the aforesaid application is relied upon and incorporated by reference in its entirety.

### FIELD OF THE INVENTION

The field of the invention relates to therapies for viral diseases. Specifically, the field of the invention relates to Aspergillus oryzae protease preparations used to treat or prevent viral diseases, including HIV and influenza.

### BACKGROUND OF THE INVENTION

HIV-1 (human immunodeficiency virus-1) infection remains a major medical problem and the number of cases of HIV and AIDS (acquired immunodeficiency syndrome) has risen rapidly. Numerous treatments for HIV are known in the art, and among other pharmaceutically active compounds, reverse transcriptase inhibitors have provided significant therapeutic effect to many HIV infected patients. For example, lamivudine (3TC) or zidovudine (AZT) are relatively well tolerated antiretroviral drugs. However, numerous viral strains have recently emerged with marked resistance against these compounds.

These drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. Despite these impressive results, 30 to 50% of patients ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present.

More recently, HIV treatment has focused on combination therapies that involve the administration of nucleoside reverse transcriptase inhibitors with protease inhibitors and with non-nucleoside reverse transcriptase inhibitors, and triple combinations of nucleoside reverse transcriptase inhibitors, non-nucleoside reverse transcriptase inhibitors and protease inhibitors. Unfortunately, combination therapies of protease inhibitors with nucleoside reverse transcriptase inhibitors are often poorly tolerated and frequently lead to premature termination of the therapy. Therefore, most current combination treatments include a combination of nucleoside reverse transcriptase inhibitors and non-nucleoside reverse transcriptase inhibitors.

Thus, there is a continuing need for new compounds and methods of treatment for HIV infection.

Influenza viruses are prevalent sources of infection in a variety of species and cause severe cold-like symptoms and can often lead to respiratory disorders and/or lethal pneumonia. Influenza viruses are classified into three types, namely types A, B, and C, on the basis of differences in the serotypes of nucleoproteins and membrane proteins. Of these, influenza virus type A and influenza virus type B are prevalent every year. The influenza type A viruses have two glycoproteins, i.e., a hemagglutinin (HA) and a neuraminidase (NA), on the surface of an envelope thereof and are thus classified into subtypes on this basis, such as H1N1, H2N2 and H3N2 on the basis of the antigenicities of the proteins. Influenza type B and influenza type C each have only one subtype.

Influenza type A viruses undergo substantial changes in antigenecity and prevail every year above other types of influenza. Antiviral agents for influenza type A viruses are known, but are not wholly satisfactory because they often cannot cope with mutations of the virus. The inability of antiviral agents to cope with the mutations of the virus is most likely due to the severity of antigenic variations of the virus.

All type A influenza viruses, including those that regularly cause seasonal epidemics of influenza in humans, are genetically labile and well-adapted to elude host defences. Influenza viruses lack mechanisms for “proofreading” and repair of errors that occur during replication. As a result of these uncorrected errors, the genetic composition of the viruses change as they replicate in humans and animals, and the existing strain is replaced with a new antigenic variant. These constant, permanent and usually small changes in the antigenic composition of influenza A viruses are known as antigenic “drift”.

The tendency of influenza viruses to undergo frequent and permanent antigenic changes necessitates constant monitoring of the global influenza situation and annual adjustments in the composition of influenza vaccines.

Influenza viruses have an additional characteristic of great public health concern. Namely, influenza type A viruses, including subtypes from different species, can swap or reassort generic materials and merge. This reassortment process, known as antigenic shift, results in novel subtypes of the virus different from both parent viruses. As populations will have no immunity to the new subtype, and as no existing vaccines can confer protection, antigenic shift has historically resulted in highly lethal pandemics of influenza. For this to happen, the novel subtype needs to have genes from human influenza viruses that make it readily transmissible from person to person for a sustainable period.

Conditions favourable for the emergence of antigenic shift have often been thought to involve humans living in close proximity of other domesticated species infected with various strain of influenza viruses. Pigs, for example, are susceptible to infection with both avian and mammalian viruses, including human strains. Thus, they can serve as a “mixing vessel” for the scrambling of genetic material from human and avian viruses, resulting in the emergence of a novel subtype. However, recently, another possible mechanism has been identified for the emergence of antigenic shift. It has been suggested that humans themselves can serve as a mixing vessel for the emergence of novel influenza subtypes.

Influenza vaccines are a popular seasonal vaccine and many people have experienced such vaccinations. However, the vaccinations are limited in their protective results because the vaccines are specific for certain subtypes of virus. The Centers for Disease Control and Prevention promote vaccination with a “flu shot” that is a vaccine that contains three influenza viruses (killed viruses): one A (H3N2) virus, one A (H1N1) virus, and one B virus. They also report that the viruses in the vaccine change each year based on international surveillance and scientists estimations about which types and strains of viruses will circulate in a given year. Thus, it is apparent that vaccinations are limited to predictions of subtypes, and the availability of a specific vaccine to that subtype.

Avian influenza virus subtype H5N1 is of particular concern for several reasons. H5N1 mutates rapidly and has a documented propensity to acquire genes from viruses infecting other animal species. Its ability to cause severe disease in humans has been documented on two occasions in Hong Kong in 1997 and 2003.

In addition, laboratory studies have demonstrated that isolates from this virus have a high pathogenicity and can cause severe disease in humans. Additionally, birds that survive infection with avian influenza subtype H5N1 excrete the virus for at least ten (10) days, thus facilitating further spread at live poultry markets and in migratory birds. The spread of infection in birds increases the opportunities for direct infection of humans. If more humans become infected over time, the likelihood increases that humans, if concurrently infected with human and avian influenza strains, could serve as the mixing vessel for the emergence of a novel subtype with sufficient human genes to be easily transmitted from person to person.

The 2009 swine flu outbreak which originated in Mexico is due to a new strain of subtype H1N1 not previously reported in pigs. In late April of 2009, the World Health Organization's Director-General declared a “public health emergency of international concern” under the rules of the WHO's new International Health Regulations when the first cases of the H1N1 virus were reported in the United States. On June 11, 2009, the Director-General of World Health Organization raised the level of influenza pandemic alert from phase 5 to phase 6.

There remains a need for effective influenza treatments, including against swine flu H1N1 that are economical and effective across numerous subtypes.

Proteolytic enzymes have been used extensively as therapeutic agents for decades. The earliest studies used pancreatic enzymes in the treatment of cancer. Later, proteolytic enzymes from non-animal sources such as the plant enzymes bromelain and papain and proteases derived from fungi such as Aspergillus sp. were investigated.

Proteases from Aspergillus oryzae are commercially used in the production of sake and soy sauce as well as in flavoring of other food products. Clinically, these enzymes have been shown to have an anti-thrombolytic/anti-hypertensive effect (Frish, E., “Clinical Review on Brinase, a Protease from Aspergillus oryzae,” Folia Haematol., 101(1):63-82 (1974), Mizuno, S., et al., “Release of Short and Proline-Rich Hypertensive Peptides from Casein Hydrolysate with an Aspergillus oryzae Protease,” J. Dairy Sci., 87:3183-3188 (2004), Sano, J., et al., “Effect of Caesin Hydrolysate Prepared with Protease Derived from Aspergillus oryzae, on Subjects with High-Normal Blood Pressure or Mild Hypertension,” J. Medicinal Food, 8(4):423-430 (2005)), anti-cancer effect (Smyth, H., et. al., “The Effects of Protease I of Aspergillus oryzae (Brinase) on Membrane Permeability and Growth of Landshutz Ascites Tumour Cells,” Int. J. cancer, 7:476-482 (1971), U.S. Pat. No. 5,562,900), and an anti-viral effect (Knight, C., “Immunogenic Properties of PR9 Influenza Virus After Treatment with Acid Protease,” Intervirology, 14:37-43(1980), Roth, R., et al., “Proteolytic Action of Aspergillus niger Extract on Influenza Virus,” Intervirology, 14:167-172 (1980), Singh, S., et al. “Isolation, Structure, and HIV-1 Integrase Inhibitory Activity of Structurally Diverse Fungal Metabolites,” J. Ind. Microbiol. Biotechnol., 30:721-731 (2003)). In addition, proteases from Aspergillus oryzae have been shown to be potent anti-inflammatory mediators (Kolodny, A., “Double Blind Evaluation of Asperkinase, a New Proteolytic Enzyme,” Am. J. Orthopedics, 234-235 (1963), U.S. Pat. No. 6,413,512 B1, U.S. Pat. No. 3,932,618, EP 1390 542).

In many diseases and injuries there is a marked increase in circulating proinflammatory cytokine levels. This increase in cytokine expression is hypothesized to contribute to the pathology of these conditions. Infection, cancer and tissue injury can all trigger the production of cytokines, which can then enter the blood stream to alter the physiology of distant tissues, or act locally as paracrine mediators. In some diseases and injury states cytokines are beneficial to the host, but in others, cytokines are detrimental to the host. Proteases and cytokines are intimately interrelated in that cytokines are involved in regulating the production of proteases and proteases are frequently involved in the liberation of soluble cytokines, as well as in their destruction. It would be beneficial therefore in certain disease states to decrease the levels of circulating cytokines. This has been accomplished by treating patients with antibodies to specific cytokines, i.e. TNFα, soluble receptor antagonists, and also proteases from plant and microbial sources.

Cytokines play a major role in the manifestation of inflammation, which is a predominant biological reaction to a myriad of injurious agents and events, including influenza and other viral diseases. It is well-known that host defensive and reparative processes in inflammation can be harmful to the body's welfare. Common characteristics of inflammation are fever, swelling, bruising and pain. The body's defensive mechanisms can bring about the release of products toxic to the host or lead to destruction of its host tissues.

Detrimental consequences of inflammation include fibrin deposition, and reduction in vascularity causing changes in tissue permeability creating additional morphologic barriers to the penetration of antibodies or pharmacological agents into the injured area. Some of the autolysis products released by tissue necrosis often constitute a good medium for microorganisms and can even antagonize the antimicrobial activity of many pharmaceutical agents, thereby exacerbating the injury or infection and prolonging the recovery process.

The absorption of orally-administered proteases in mammals has been extensively studied. The prevailing finding of these studies is that proteases can be partially absorbed intact, with activity preserved, from the digestive tract and subsequently distributed systemically in the blood. Proteolytic enzymes from Aspergillus oryzae are often used as digestive aids, and as such stimulate bowel movements, often times leading to diarrhea in the host.

Early in the study of proteases, it was observed that the administration of animal-derived proteases could accelerate the healing of inflamed sites. Therefore, a large database exists of clinical results from orally-administered, animal derived proteases establishing the effectiveness of these proteases as therapeutic agents for inflammatory conditions. However, a clear mechanism of physiological action for animal-derived proteases is yet to be determined. Plant proteases have also been, found to have a positive effect on inflammation The largest body of evidence supporting the use of proteases for inflammatory conditions studied the effects of a mixture of papain, bromelain, trypsin, chymotrypsin, pancreatin and rutin. In most cases, the mixture was in addition to standard medical care.

It has long been established that a number of chemical compounds typically referred to as vitamins and minerals provide significant health value and treat specific medical conditions, particularly when supplied in therapeutic doses. Over the years, a number of such vitamins and minerals have been identified. For example, vitamins include A, C, D, E, and the family of B vitamins and minerals include iron, zinc, calcium and chromium. The human body does not synthesize most of these vitamins and minerals which are essential to maintaining the health of the human body. Thus these necessary vitamins and minerals must be obtained from an external source. The two most common external sources are foods and nutritional supplements. Food is typically the primary source of obtaining the necessary nutrients for maintaining health, however many people do not eat foods that consistently provide the necessary daily requirements of vitamins and minerals. Thus, vitamin and mineral nutritional supplementation has become a recognized method of meeting these daily requirements.

While certain vitamins and minerals have been shown to be essential for the maintenance of an individual's health, the use of vitamin and mineral nutritional supplementation has afforded the possibility to include micro-nutrients which, although not absolutely essential to maintaining health, provide significant benefit toward maintaining health. U.S. Pat. No. 6,413,512 Bi describes treating patients suffering from a disease resulting from increased cytokine production with a pharmaceutical composition comprising 2 or more proteases from a microbial source in an amount of between 20,000 HUT and 550,000 HUT. The protease described in this patent is made using rice and/or wheat bran as a carbohydrate source. Prior researchers have shown activity of proteolytic enzymes against viral protein capsids in vitro. However, when attempts were made to establish the safety and efficacy of proteases against viruses in vivo, the following problems were encountered: Although the enzymes had a pH of optimum activity, most also had a pH of optimum stability; unfortunately, in many cases, the pH of optimum stability was far removed from the pH of optimum activity and final formulation for application required the use of buffer solutions to bring the enzyme from a pH of optimum stability to that of optimum activity which was rarely, if ever done; when the enzymes were taken orally, they were digested in the gastrointestinal tract and rarely reached the bloodstream in effective concentrations; both intramuscular and intravenous administrations were tried but with many adverse reactions to repeated injections that did not encourage patient compliance; injectable use was virtually abandoned; the effectiveness of individual natural enzymes was never established, and mixtures of these natural enzymes were often tried unsuccessfully. Since the effectiveness, and thus proper dosage level for any of the natural enzymes was never established, most of the trials of these mixtures were run using sub-threshold dosage levels. Further, the present invention involves the application of a highly purified and stable anti-inflammatory proteolytic enzyme significantly having the ‘pH’ of optimum stability essentially equal to the ‘pH’ of optimum activity.

In light of the above, the present invention is based on the surprising result that a protease composition from Aspergillus oryzae complexed with potato dextrin as a resistant starch colloidal carrier is better absorbed by the proximal small intestine and is effective as an antiviral agent, particularly against HIV, influenza and hepatitis C infection. Proteases complexed with potato dextrin as a resistant starch colloidal carrier, in contrast to those which contain rice and or wheat bran, reduce gastrointestinal side effects such as diarrhea. In addition, administering more than 2,000,000 HUT/day of Aspergillus oryzae protease complexed with potato dextrin, as a resistant starch colloidal carrier provides an optimal and therapeutic anti-inflammatory and antiviral effect, particularly against HIV, influenza, and hepatitis C.

### BRIEF SUMMARY OF THE INVENTION

In one aspect, the invention provides a method of treating HIV infection in a subject in need thereof, comprising orally administering to said subject a composition comprising a therapeutically effective amount of an Aspergillus oryzae protease and potato dextrin as a resistant starch colloidal carrier.

In another aspect, the invention provides a method of treating influenza infection in a subject in need thereof, comprising orally administering to said subject a composition comprising a therapeutically effective amount of an Aspergillus oryzae protease and potato dextrin as a resistant starch colloidal carrier.

In some embodiments, the amount administered is about 2,000,000 HUT per day or more.

In another aspect, the methods of the invention further comprise administering nutritional supplement of vitamins and minerals. In some embodiments, the nutritional supplement composition comprises vitamin A, vitamin B1, vitamin B2, vitamin B5, vitamin B6, vitamin B 12, vitamin C, magnesium citrate, vitamin E, Vitamin D3, zinc citrate, manganese gluconate, copper gluconate, biotin, folate, chromium polynicotinate, citrus bioflavinoids, glucosamine sulfate, and boron sulfate. In some embodiments, the nutritional supplement is administered 2-3 times per day with food while the protease composition is given 4 times daily on an empty stomach.

### DETAILED DESCRIPTION OF THE INVENTION

It is to be understood that this invention is not limited to the particular methods, compositions and materials disclosed herein as such methods, compositions and materials may vary. It is also understood that the terminology employed herein is used for the purpose of describing particular embodiments only and is not intended to be limiting since the scope of the present invention will be limited only by the appended claims and equivalents thereof

It must also be noted that, as used in this specification and the appended claims, the singular forms “a,” “an,” and “the” include plural referents unless the context clearly dictates otherwise. Thus, for example, reference to “a protease” includes references to two or more of such proteases and “a vitamin” includes reference to one or more of such vitamins, unless otherwise specified.

As used herein, “treat” and all its forms and tenses (including, for example, treating, treated, and treatment) can refer to therapeutic or prophylactic treatment. In certain aspects of the invention, those in need thereof of treatment include those already with a pathological condition of the invention (including, for example, HIV or influenza infection), in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the pathological condition. In other certain aspects of the invention, those in need thereof of treatment include, those in which a pathological condition is to be prevented, in which case treating refers to administering a therapeutically effective amount of a composition to a subject (including, for example, a human or other mammal in need of treatment) at risk of developing the pathological condition, including HIV or influenza infection.

The term “administration” and variants thereof (e.g., “administering” a composition or preparation) in reference to a composition or preparation of the invention mean providing the composition or preparation to the individual, in need of treatment. When a composition or preparation of the invention is provided in combination with one or more other active agents (e.g., antiviral agents useful for treating HIV infection, AIDS, or influenza infection), “administration” and its variants are each understood to include concurrent and sequential provision of the composition or preparation and other agents.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” or “therapeutically effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented.

In some embodiments, the present invention provides a method of treating viral diseases by administering a therapeutically effective amount of a protease preparation derived from Aspergillus oryzae complexed with potato dextrin as a resistant starch colloidal carrier.

In some embodiments, the amount of the Aspergillus oryzae protease administered is more than about 2,000,000 HUT per day. Greater amounts can also be administered, including about 3,000,000, about 5,000,000, about 7,000,000, about 10,000,000, about 15,000,000, about 25,000,000 or about 30,000,000 HUT per day. In some embodiments, the amount administered ranges from about 2,000,000 HUT/day to about 25,000,000 HUT/day. In some embodiments, the amount administered ranges from about 3,000,000 HUT/day to about 15,000,000 HUT/day, from about 4,000,000 HUT/day to about 10,000,000 HUT/day, from about 5,000,000 HUT/day to about 7,500,000 HUT/day.

The composition can be administered once per day, or more than once per day. In some embodiments, the protease preparation is given on an empty stomach four times daily. In some embodiments, it is administered 2, 3, 4, 5, 6, or 7 times or more per day.

Another embodiment of the invention provides a method of treating viral infection such as HIV or influenza by administering a protease preparation derived from Aspergillus oryzae complexed with potato dextrin as a resistant starch colloidal carrier at an amount of more than about 2,000,000 HUT per day, in combination with a nutritional supplement of vitamins and minerals. The nutritional supplement preferably contains vitamin A, vitamin B1, vitamin B2, vitamin B5, vitamin B6, vitamin B12, vitamin C, magnesium citrate, vitamin E, Vitamin D3, zinc citrate, manganese gluconate, copper gluconate, biotin, folate, chromium polynicotinate, citrus bioflavinoids, glucosamine sulfate, and boron sulfate. The nutritional supplements are preferably administered sequentially (either before or after) with the protease composition and not concurrently. In some embodiments, the protease composition is administered first on an empty stomach, and the nutritional supplements are administered later with food, after the enzyme has traversed the stomach and has been absorbed by the intestines. In some embodiments, the nutritional supplement is given 2-3 times per day with food while the protease preparation is given 4 times daily on an empty stomach.

The present invention offers improvements over prior proteolytic products in that, unlike other protease compositions, the primary benefit is obtained from the use of a protease from a particular microbial source in a defined dosing regimen. In some embodiments, the invention does not contain animal-derived products, and thus is acceptable to patients who may object to the ingestion of animal products. The invention focuses specifically on protease preparations complexed with potato dextrin as a resistant starch colloidal carrier instead of maltodextrin, wheat or rice bran. Surprisingly, protease preparations prepared using potato dextrin are more readily absorbed by the proximal small intestine and lead to less undesirable gastrointestinal side effects than those prepared with maltodextrin, wheat or rice bran. In addition, in some embodiments, the invention does not contain gluten and may be safely ingested by persons who have an allergy to wheat gluten.

In some embodiments, the Aspergillus oryzae protease preparation complexed with potato dextrin as a resistant starch colloidal carrier is Protease A-DS (obtained from Amano Enzyme U.S.A. Co., Ltd., Elgin, Ill.). This enzyme preparation contains not less than 300,000 HUT/gram.

The protease preparation can be orally administered in capsule (hard or soft), tablet (coated or uncoated), powder or granule (coated or uncoated) or liquid (solution or suspension) form. In some embodiments, the protease preparation can be given dissolved or suspended in water. In some embodiments, the protease preparation is provided in capsular form using standard methods and techniques.

In some embodiments, the viral disease is selected from the group consisting of influenza, human immunodeficiency virus (HIV), Hepatitis A, B, C, D or E, Ebola and dengue fever. Treatments of acute and chronic versions of the diseases are contemplated by the present invention.

In one embodiment, the viral disease is HIV. In some embodiments, the composition is administered in combination with an HIV therapeutic agent selected from the group consisting of one or more HIV reverse transcriptase inhibitor(s), a HIV protease inhibitor and combinations thereof. The reverse transcriptase inhibitor can be a nucleoside analog reverse transcriptase inhibitor, nucleotide analog reverse transcriptase inhibitor, or a non-nucleoside reverse transcriptase inhibitor. Exemplary nucleoside analog reverse transcriptase inhibitors include zidovudine (RETROVIR), didanosine (VIDEX), stavudine (ZERIT), lamivudine (EPIVIR), abacavir (ZIAGEN), emtricitabine (EMTRIVA), entecavir (BARACLUDE) and apricitabine. Nucleotide analog reverse transcriptase inhibitors include tenofovir (VIREAD) and adefovir (PREVEON and HEPSERA). Non-nucleoside reverse transcriptase inhibitors include efavirenz (SUSTIVA and STOCRIN), nevirapine, nevirapine (VIRAMUNE), delavirdine (RESCRIPTOR) and etravirine (INTELENCE). Exemplary HIV protease inhibitors include saquinavir, ritonavir, indinavir, nelfinavir, and amprenavir.

In some embodiments, the viral disease is influenza. In some embodiments, the influenza is selected from the group consisting of swine flu, avian flu, Brisbane flu, and seasonal flu strains. In some embodiments, the influenza strain is the swine flu strain H1N1 which originated in Mexico and is responsible for the 2009 outbreak. In some embodiments, the influenza strain is an avian influenza strain selected from the group consisting of H5N1, H7N7, H7N3, H9N2 and H7N2. In some embodiments, the influenza strain is the Brisbane flu. In some embodiments, the Brisbane flu strain is selected from the group consisting of H3N2 and H1N1. In some embodiments, the influenza strain is seasonal flu. In some embodiments, the seasonal flu strain is H3N2. In some embodiments the flu strain is H 1N2, H2N2, H5N2, H5N8, H5N9 or H7N3.

In accordance with the invention, the influenza virus can be of any hemagglutinin type, including H1, H2, H3, H4, H5, H6, H7, H8, H9, H10, H11, H12, H13, H14, H15 or H16. The influenza virus can be of any neuraminidase type, including N1, N2, N3, N4, N5, N6, N7, N8 or N9. Thus, the influenza virus can be any combination of hemagglutinin and neuraminidase types.

In another embodiment an additional supplement of calcium is administered. In some embodiments, calcium is administered at a dose of 900 mg/day. In some embodiments, the additional calcium supplement is administered once daily in the evening.

In one embodiment, the nutritional supplement of the invention comprises vitamin A, vitamin B1, vitamin B2, vitamin B5, vitamin B6, vitamin B12, vitamin C, magnesium citrate, vitamin E, vitamin D3, zinc citrate, manganese gluconate, copper gluconate, biotin, folate, chromium polynicotinate, citrus bioflavinoids, glucosamine sulfate, and boron sulfate.

In one embodiment, the dietary supplement comprises vitamin A. Vitamin A functions as a regulatory hormone with effects on specific genes for differentiation and maintenance of epithelial tissue, and is important to reproduction, vision and immune function. Taken in excess vitamin A will cause birth defects, and in the active or athletically performing individual, can cause bone demineralization, loss of elasticity in connective tissue, muscle soreness and joint pain. The recommended daily allowance (RDA) for vitamin A is 5000 IU/day. In some embodiments of the invention the dietary supplement contains 5000 IU/day of vitamin A in the form of retinyl palmitate.

In one embodiment, the dietary supplement comprises vitamin B1. Vitamin B1 is distributed widely in foods in low concentrations. Vitamin B1 is susceptible to destruction by refining processes, neutral and alkaline conditions, heat and oxidation. Vitamin B 1 is important in energy production from food, especially carbohydrates, and plays a vital role in nerve function. Supplementation in large doses is safe and has shown some efficacy in the ability to control pain in connective tissue. The RDA for vitamin B1 is 1.5 mg/day. In some embodiments the dietary supplement contains 100 mg/day in the form of thiamin mononitrate.

In one embodiment, the dietary supplement comprises vitamin B2. Vitamin B2 is essential to a large number of redox reactions, releasing energy from carbohydrates, fats and amino acids, and is thus important to the elderly, and the active or athletic individual. Vitamin B2 is highly water soluble and reactive to light. Strict vegetarians, pregnant and lactating women, and ill or trauma victims are also at risk for vitamin B2 deficiency. High oral doses of vitamin B2 are essentially non-toxic. The RDA for vitamin B2 is 1-1.5 mg/day. In some embodiments the dietary supplement contains 50 mg/day vitamin B2.

In one embodiment, the dietary supplement comprises vitamin B3 (niacinamide, not niacin). Vitamin B3 plays an important role in energy production, cellular respiration, fat synthesis and joint pain and mobility. Vitamin B3 (niacinamide, not niacin) possesses no known side effects, and when supplemented several times during the day, demonstrates long-lasting objective improvements in joint mobility. The RDA for vitamin B3 (niacin) is 15-20 mg/day. In some embodiments the dietary supplement contains 300 mg/day vitamin B3 (niacinamide).

In one embodiment, the dietary supplement comprises vitamin B5. Vitamin B5 plays a significant role in energy production from carbohydrates, fats and proteins. The toxicity of vitamin B5 is negligible. Active and elderly individuals in trauma or who suffer from rheumatoid arthritis have realized significant improvements in morning stiffness, disability and pain when supplemented with gram doses of vitamin B5. There is no RDA for vitamin B5, a provisional range of intake of 4-87 mg/day was established 1980. In some embodiments the dietary supplement contains 1000 mg/day vitamin B5 in the form of panthothenic acid.

In one embodiment, the dietary supplement comprises vitamin B6. Vitamin B6 has recently been shown to be vital to bone health. However in high doses, vitamin B6 can be toxic. In low doses, vitamin B6 shows no efficacy. Pregnant and lactating women, oral contraceptive users and heavy drinkers are at risk for vitamin B6 deficiency. The RDA for vitamin B6 is 1.5-2 mg/day. In some embodiments the dietary supplement contains 50 mg/day vitamin B6 in the form of pyridoxine hydrochloride.

In one embodiment, the dietary supplement comprises vitamin B12. Vitamin B12 deficiencies can interfere with normal cell division involving arrested synthesis of DNA causing cellular mutations leading to disease states, particularly in bone marrow and intestinal mucosa. Vitamin B12 has no appreciable toxicity, and is frequently deficient in strict vegetarians. The RDA for vitamin B12 is 2 μg/day. In some embodiments the dietary supplement contains 100 μg/day vitamin B12.

In one embodiment, the dietary supplement comprises vitamin C. Most of the functions of vitamin C are directly applicable to the health of connective tissue and their response to injury. Vitamin C, however tends to change the valence of copper, rendering copper unavailable to the body. It is good nutritional practice to dose extra copper when using mega doses of vitamin C. The RDA for vitamin C is 50-60 mg/day. In some embodiments the dietary supplement contains 500 mg/day vitamin C.

In one embodiment, the dietary supplement comprises magnesium. Magnesium is widely distributed in food. However, the refining and processing of foods tends to remove large amounts of magnesium. Magnesium fulfills so many essential functions that it is almost impossible to single out any one function as most critical. There is no established RDA for magnesium because it is ubiquitous in nature. Nonetheless, the food and nutrition board of the national academy of sciences has recommended intake based on age and gender of 40-400 mg/day as safe and adequate. In some embodiments the dietary supplement contains 400 mg/day magnesium in the form of magnesium citrate.

In one embodiment, the dietary supplement comprises vitamin E. Vitamin E is synthesized only by plants, and therefore is found primarily in plant products, particularly in plant oils. Vitamin E affects almost every aspect of health to some degree in its role and function as a scavenger of free radicals. The RDA for vitamin E is 8-10 mg/day. In some embodiments the dietary supplement contains 400 mg/day vitamin E.

In one embodiment, the dietary supplement comprises vitamin D3. Vitamin D3 is important in calcium, phosphate and magnesium absorption. Excess vitamin D causes hypercalcemia. Clinical signs are weakness, nausea, headaches, abdominal pain, cramps and diarrhea. Intake of vitamin D is not absolutely essential if adequate skin exposure to sunlight is available. The RDA for vitamin D3 is 400 IU/day. However, recent research has shown that the recommended dose of vitamin D3 should be 1000-2000 IU/day due to the newly discovered multiplicity of critical functions in metabolism other than simply bone health. In some embodiments the dietary supplement contains 1000 IU/day vitamin D3.

In one embodiment, the dietary supplement comprises zinc. Zinc, in addition to cell growth and replication, has specific roles in sexual maturation, fertility, reproduction, night vision, immune function, taste and appetite. Zinc, with copper as a stabilizing influence, is vital to genetic stability and expression during cellular replication. Deficiencies or excesses of zinc can result in mutated cellular replication leading to disease states. Thus, zinc should be supplemented in balance with copper to protect the cellular reproductive function. The RDA for zinc is 12-15 mg/day. In some embodiments the dietary supplement contains 25 mg/day of zinc in the form of zinc citrate.

In one embodiment, the dietary supplement comprises manganese. Manganese plays unique and vital roles in the synthesis of macromolecular components of connective tissues, especially for bone and cartilage. Since acute, severe deficiencies of manganese are rare, defects of manganese status appear to occur in active individuals during periods of stress, or from a life-long, chronic, intermittent, or marginal deficiency. Acute deficiency symptoms are not usually encountered but rather, as with copper and zinc, chronic or marginal deficiencies in manganese uptake results in decreased synthesis of connective tissues leading to loss of integrity for joints and bones. The RDA for manganese is 2.0 mg/day. In some embodiments the dietary supplement contains 10 mg/day of manganese in the form of manganese gluconate.

in one embodiment, the dietary supplement comprises copper. Modest doses of copper as organic chelates are used to maintain physiologic levels of cuproenzymes important to connective tissue, particularly in the athletic or active individual. Copper has a long history of medicinal uses, including treatment of inflammatory conditions, osteoporosis, and arthritis. Copper functions primarily as a component of metalloenzymes with essential functions, and also activates other enzymes. There is no RDA for copper. Current research however, has established a beneficial, safe and adequate intake of 2-8 mg/day. In some embodiments the dietary supplement contains 8 mg/day of copper in the form of copper gluconate.

In one embodiment, the dietary supplement comprises biotin. Biotin is important for energy production and fat metabolism. Biotin is rather widespread among foods and is synthesized by intestinal flora. Simple deficiencies of biotin in humans in the absence of other nutrient deficiencies are rare. However, those at risk for biotin deficiency include individuals on antibiotic therapy, alcoholics, pregnant and lactating women, surgical burn patients and the elderly. Relatively low levels of biotin have also been reported in physically active or athletic individuals. There is no RDA for biotin. However, the national academy of sciences food and nutrition board has published a nominal safe and adequate intake of 100-200 μg/day. In some embodiments the dietary supplement contains 1000 μg/day of biotin.

In one embodiment, the dietary supplement comprises folate. Folate is important to blood cell formation as well as DNA and RNA synthesis. Deficiencies result in reduced cell division which is manifested as anemia, skin lesions and poor overall growth. Pregnant and lactating women, elderly persons and those taking certain folate antagonists such as aspirin, have an increased requirement for folate in the diet. The RDA for folate is 150-200 μg/day. In some embodiments the dietary supplement contains 1000 μg/day of folate.

in one embodiment, the dietary supplement comprises chromium. Chromium is essential for optimal peripheral insulin action with respect to glucose intake. Studies of elderly and active adults with noninsulin-dependent diabetes mellitus showed improvement in glucose tolerance following a period of chromium supplementation. The RDA for chromium is 120 μg/day. In some embodiments the dietary supplement contains 200 μg/day of chromium in the form of chromium polynicotinate.

In one embodiment, the dietary supplement comprises bioflavinoids. Bioflavinoids are a ubiquitous class of compounds found in plants. Most bioflavinoids exhibit antioxidant activity. Scavenging hydroxyl radicals, lipid peroxides, and reactive oxygen species has been repeatedly documented for many bioflavinoids. Bioflavinoids also reduce capillary fragility and/or permeability. This effect “spares” vitamin C, and is likely due to flavinoid chelation and antioxidant properties, particularly important to the physically active or elderly individual. Bioflavinoids appear to render other nutrients more effective as anti-inflammatory agents, especially vitamin C and proteolytic enzymes. There is no RDA for bioflavinoids. In some embodiments the dietary supplement contains 1000 mg/day of bioflavinoids in the form of citrus bioflavinoids.

In one embodiment, the dietary supplement comprises glucosamine. Glucosamine is a naturally occurring amino sugar found in glycoproteins and glycosaminoglycans. Increased availability of glucosamine through supplements accelerates or enhances synthesis of hyaluronan, glycosaminoglycans and proteolysis. There is no RDA for glucosamine. In some embodiments the dietary supplement contains 1000 mg/day of glucosamine in the form of glucosamine sulfate.

In one embodiment, the dietary supplement comprises boron. Maintenance of boron intake by dietary manipulation and/or supplementation is recommended for bone loss conditions such as osteoporosis, fracture healing, arthritis and other degenerative joint diseases. There is no RDA for boron; however research has indicated that a boron intake of 3-6 mg/day is beneficial, safe and adequate. In some embodiments the dietary supplement contains 3 mg/day of boron in the form of boron citrate.

In one embodiment, the dietary supplement is taken with an additional calcium supplement. Calcium should be given as a single dose, once per day in the evening. Calcium requirements should be provided by dietary means first. When increasing calcium intake through supplemental means to reach the recommended levels of 800-1200 mg/day, doses of 900 mg elemental calcium should be taken once daily with the evening meal. Supplementing calcium in the evening is preferred because calcium metabolizes differently in the early evening and is better absorbed at that time.

The present invention will be further illustrated by the following examples that are not limited. The present invention may be better understood by reference to the following non-limiting Examples, which are provided as exemplary of the invention. The following examples are presented in order to more fully illustrate some embodiments of the invention. They should in no way be construed, however, as limiting the broad scope of the invention.

### EXAMPLES

### Example 1

A suitable Aspergillus oryzae protease preparation complexed with potato dextrin as a resistant starch colloidal carrier is Protease A-DS, obtained from Amano Enzyme U.S.A. Co., Ltd., Elgin, Ill. This enzyme preparation contains not less than 300,000 HUT/gram. The protease extract can be given dissolved or suspended in water or in capsular form.

The protease preparation can be administered sequentially with the nutritional supplements shown below. For example, the nutritional supplements can be administered with food 1 hour after administration of the enzyme, to ensure the enzyme is administered on an empty stomach.

| | AMOUNT PER SERVING | |
|---|---|---|
| VITAMIN/MINERAL | (serving size 9 capsules) | |
| Vitamin A (as retinyl palmitate) | 5,000 | IU |
| Vitamin B1 (as thiamin mononitrate) | 100 | mg |
| Vitamin B2 (as riboflavin) | 50 | mg |
| Vitamin B3 (as niaciniaminde) | 300 | mg |
| Vitamin B5 (as pantothenic acid) | 1,000 | mg |
| Vitamin B6 (as pyridoxine hydrochloride) | 50 | mg |
| Vitamin B12 (as cyanocobalamin) | 100 | mcg |
| Vitamin C (as ascorbic acid) | 500 | mg |
| Magnesium citrate | 400 | mg |
| Vitamin E (as d-alpha-tocopherol) | 400 | IU |
| Vitamin D3 (as cholecalciferol) | 1000 | IU |
| Zinc Citrate | 25 | mg |
| Manganese Gluconate | 10 | mg |
| Copper Gluconate | 8 | mg |
| Biotin (as d-biotin FCC) | 1 | mg |
| Folate (as folic acid) | 1 | mg |
| Chromium polynicotinate | 200 | mcg |
| Citrus bioflavinoids | 1,000 | mg |
| Glucosamine Sulfate (13.2% potassium) | 1,000 | mg |
| Boron Citrate | 3 | mg |

### Example 2

An adult male patient co-inflected with HIV and hepatitis C was treated with a proteolytic enzyme in accordance with the methods of the present invention. The patient was administered 6 grams (1,800,000 HUT) of Protease A-DS dissolved in water 3 times per day on an empty stomach for a total daily dose of approximately 5,400,000 HUT.

Ten days following treatment, he exhibited a Hepatitis C RNA level of 8,070,000 IU/ml. Within five weeks of treatment thereafter, his Hepatitis C RNA level had dropped to about 3,600,000 IU/ml.

### Example 3

A 19 year-old woman was suffering from an apparent infection by Brisbane influenza. On day 1, she complained of a mild sore throat, general malaise, and mild dizziness. That evening, she exhibited restless behavior and had difficulty sleeping. On day 2, her throat had become progressively worse, as noted by her discomfort, and she exhibited nasal and chest congestion accompanied by some coughing. By early afternoon (˜2:00 p.m.), she complained of non-specific body aches and pains, an inability to drive, and impaired cognitive ability to read, write, or even to watch television. It was taxing for her to try to comprehend and respond to oral communication. She became dizzy when standing and had difficulty walking around and climbing stairs. At around 2:30 p.m., one dose of the proteolytic enzyme (Protease A-DS) according to the claimed invention was administered (1,800,000 HUT). Three capsules of the nutritional supplements of example 1 were administered with a light meal one-half hour after the enzyme was given. At about 6:30 p.m., the patient exhibited hot and cold flashes, but also was releasing some of the congestion via nasal mucous discharge and coughing. She went to bed, and was given another dose of the proteolytic enzyme (Protease A-DS; 1,800,000 HUT) followed 45 minutes later with some healthy food and 3 capsules orally of the nutritional supplements,

By 9:00 p.m., she was able to watch television while lying down and engage in simple conversation. The hot and cold flashes continued at a reduced level, and the patient felt better in general.

On day 3, a further dose of the proteolytic enzyme (1,800,000 HUT) was administered late morning (around 10 am). Three capsules of the nutritional supplements were given during her light meal around 11:30 a.m. Although the hot and cold flashes continued (predominantly periods of feeling cold) intermittently, the patient's mental clarity and focus improved significantly by the afternoon. Considering the symptoms and severity of her illness at its worst her fever was fairly low (a maximum of 101.2 degrees) and she rested all day. She also had continuing nasal discharge and coughing. Around 5:30 p.m., she a further dose of the proteolytic enzyme (1,800,000 HUT). Around 6:30 p.m., she took 3 capsules of the nutritional supplements with her dinner and she slept well that evening.

On the morning of day 4, the patient was back to feeling her customary self and she resumed her daily activities. She still had a residual cough and some nasal discharge, but the other symptoms were completely gone. She continued precautionary and preventive treatment measures with the proteolytic enzyme (Protease A-DS; 1,800,000 HUT per dose) and the Nutritional Supplements (3 capsules at mealtimes) and she began a regimen of three times per day for both.

### Example 4

An adult male patient infected with HIV was treated with a protease enzyme from Aspergillus oryzae in accordance with the methods of the present invention during a three week period. The patient was administered 6 grams (1,800,000 HUT per dose) of Protease A-DS dissolved in water 3 times per day on an empty stomach for a total daily dose of approximately 5,400,000 HUT. The patient discontinued his normal HIV medication and nutritional supplements for six weeks prior to administration of the proteolytic enzyme. The patient's HIV viral load and absolute CD4 helper cell count was assayed immediately prior to treatment with the protease composition and following the three week treatment period. The patient's HIV viral count increased from 62910 copies/mL to 77070 copies/mL (an increase of 22%) during the course of treatment. Surprisingly, the patient's absolute CD 4 helper count rose from a low value of 321 cells per μL (below the normal range) to 485 cells per μL (within the normal range) at the conclusion of the three week period, an increase of 51%.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

I claim:

1. A method of treating HIV infection in a subject in need thereof, comprising orally administering to said subject a composition comprising a therapeutically effective amount of an Aspergillus oryzae protease and potato dextrin as a resistant starch colloidal carrier.

2. The method of claim 1, wherein the Aspergillus oryzae protease is administered in an amount greater than about 2,000,000 HUT per day.

3. The method according to claim 1, wherein the composition is administered on an empty stomach.

4. The method according to claim 1, wherein the composition is administered 4 times per day.

5. The method of claim 1, wherein the composition is in liquid form.

6. The method of claim 1, further comprising administering a nutritional supplement comprising vitamins and minerals.

7. The method according to claim 1, wherein the composition is administered in combination with an HIV therapeutic agent selected from the group consisting of one or more HIV reverse transcriptase inhibitor(s), a HIV protease inhibitor and combinations thereof.

8. A method of treating an influenza infection in a subject in need thereof, comprising orally administering to the subject a composition comprising a therapeutically effective amount of an Aspergillus oryzae protease and potato dextrin as a resistant starch colloidal carrier.

9. The method of claim 8, wherein the Aspergillus oryzae protease is administered in an amount greater than about 2,000,000 HUT per day.

10. The method according to claim 8, wherein the composition is administered on an empty stomach.

11. The method according to claim 8, wherein the composition is administered 4 times per day.

12. The method of claim 8, wherein the composition is in liquid form.

13. The method of claim 8, further comprising administering a nutritional supplement comprising vitamins and minerals.

14. The method according to claim 13, wherein the nutritional supplement comprises vitamin A, vitamin B1, vitamin B2, vitamin B5, vitamin B6, vitamin B12, vitamin C, magnesium citrate, vitamin E, vitamin D3, zinc citrate, manganese gluconate, copper gluconate, copper gluconate, biotin, folate, chromium polynicotinate, citrus bioflavinoids, glucosamine sulfate and boron sulfate.

15. The method of claim 14, wherein the amount of vitamin A administered per day is from 3500-7500 IU, the amount of vitamin B1 administered per day is from 35-250 mg, the amount of vitamin B2 administered per day is from 15-85 mg, the amount of vitamin B5 administered per day is from 500-1500 mg, the amount of vitamin B6 administered per day is from 10-100 mg, the amount of vitamin B12 administered per day is from 25-250 mcg, the amount of vitamin C administered per day is from 175-950 mg, the amount of magnesium citrate administered per day is from 100-800 mg, the amount of vitamin E administered per day is from 150-500 IU, the amount of vitamin D3 administered per day is from 750-1500 IU, the amount of zinc citrate administered per day is from 10-75 mg, the amount of manganese gluconate administered per day is from 1-20 mg, the amount of copper gluconate administered per day is from 2-25 mg, the amount of biotin administered per day is from 0.2-3.0 mg, the amount of folate administered per day is from 0.2-3.0 mg, the amount of chromium polynicotinate administered per day is from 50-400 mcg, the amount of citrus bioflavinoid administered per day is from 500-1500 mg, the amount of glucosamine sulfate administered per day is from 750-1500 mg and the amount of boron sulfate administered per day is from 1-10 mg.

16. The method according to claim 14, wherein the nutritional supplements are administered 2-3 times per day.

17. The method according to claim 15, wherein the nutritional supplements are administered with food.

18. The method according to claim 8, wherein the influenza is selected from the group consisting of avian flu, swine flu, Brisbane flu and seasonal flu.

19. The method according to claim 8, wherein an additional dietary supplement of calcium is administered.

20. The method of claim 19, wherein calcium is given at a dose of 900 mg/day.
